(19) United States
(12) Patent Application Publication
(10) Pub. No.: US 2013/0190774 A1
(43) Pub. Date: Jul. 25, 2013

(54) MECHANICAL POSITIONING SYSTEM FOR SURGICAL INSTRUMENTS

(71) Applicants: Ricardo Beira, Lausanne (CH); Clavel Reymond, Oulens-sous-Echallens (CH); Hannes Bleuler, Buchillon (CH)

(72) Inventors: Ricardo Beira, Lausanne (CH); Clavel Reymond, Oulens-sous-Echallens (CH); Hannes Bleuler, Buchillon (CH)

(73) Assignee: ECOLE POLYTECHNIQUE FERDERALE DE LAUSANNE (EPFL), Lausanne (CH)

(21) Appl. No.: 13/816,324

(22) PCT Filed: Aug. 11, 2011
(86) PCT No.: PCT/IB2011/053576

§ 371 Date: Mar. 14, 2013

(30) Foreign Application Priority Data
Aug. 11, 2010 (EP) 10172517.4

(51) Int. Cl. A61B 19/00

(52) U.S. Cl. CPC A61B 19/22
USPC 606/130

(57) ABSTRACT

An external manipulator for positioning surgical instruments within the abdominal cavity, comprising a hybrid kinematics with a parallel structure, able to provide four, active or passive, positional degrees of freedom to a endoscopic unit, placed in the distal end of an instrument shaft. Due to this specific kinematics, the instrument shaft is able to perform two rotations, one translation, and a fourth orientation degree of freedom about a remote centre of rotation, coincident with the surgical incision port. Because of its unique design and kinematics, the proposed mechanism is highly compact, stiff and its dexterity fulfils the workspace specifications for surgical procedures.

### FIELD OF THE INVENTION

The present invention concerns an external manipulator for positioning surgical instruments within the abdominal cavity. More specifically, the manipulator comprises a hybrid kinematics with a parallel structure, able to provide four, active or passive, positional degrees of freedom to a endoscopic unit, placed in the distal end of an instrument shaft.

### BACKGROUND OF THE INVENTION

A major progress in abdominal surgery has occurred during the last decades with the introduction of laparoscopic and minimally invasive techniques. These innovative procedures focused much attention due to their several advantages: smaller abdominal incisions needed, resulting in faster recovery of the patient, improved cosmetics, and shorter stay in the hospital. The safety, efficiency and cost-effectiveness of laparoscopic surgery have subsequently been demonstrated in clinical trials for many routine abdominal operations. However, from the surgeon's point of view there are still many difficulties in learning and performing such procedures with current laparoscopic equipment, which is non-ergonomic, non-intuitive and missing in adequate visual and tactile feedback.

In order to overcome the disadvantages of traditional minimally invasive surgery (MIS), robot technology has been introduced into the operation room. Although a wide range of diagnostic and therapeutic robotic devices have been developed, the only commercial systems that have already been used in human surgery are the da Vinci System, by Intuitive Surgical and ZEUS, by Computer Motion. Following the fusion between the two companies, the ZEUS robot is no longer produced.

Despite various existing interesting systems and after more than ten years of robotic MIS research, the surgical robotics field is still only at the very beginning of a very promising large scale development. One of the major open drawbacks is that the current surgical robots are voluminous, competing for precious space within the operating room (OR) environment and significantly increasing ORs preparation time. Access to the patient is thus impaired and this raises safety concerns. In addition, although robotic systems offer excellent vision and precise tissue manipulation within a defined area, they are limited in operations involving more than one quadrant of the abdomen. Since many gastrointestinal operations involve operating in at least two abdominal quadrants, the repeated disconnection and movement of the robots increase significantly the duration of the surgical procedure.

Another drawback of current surgical systems is related to the mechanisms that hold and place the surgical instruments into the abdomen, remaining external to the patient. Their access within the abdomen is limited since the instruments are constrained by the abdominal wall at their point of entry. They are also further restricted by this fulcrum effect, due to the fact that their internal motion is mirrored and magnified outside the body by the robotic arms. Moreover, surgical instruments have limited space to move, leading to eventual collisions. Consequently, a key feature of most surgical robot systems is a remote center of rotation (RCM) that allows the manipulated tool to pivot around a fixed point, coincident with the insertion point of the tool through the patient's abdomen. Although some robotic systems use a passive RCM, or a virtually constrained RCM, a mechanically constrained RCM is often considered safer.

There is a variety of ways to achieve a mechanically constrained RCM. For those spherical mechanisms comprising revolute joints, all the rotation axes intersect at the centre of the mechanism, effectively meeting the pivot constraint of MIS. The implementation of this approach can be done using parallelogram linkages, or other linkage configuration. However, in most cases, actuators are directly mounted at the joints. This may result in: (1) addition of inertial loads that adversely affect system performance or (2) lack of DOEs to place and guide the MIS instruments within the abdominal cavity.

Although some robotic systems use a passive RCM [Sackier1994], [Cavusoglu1999], or a virtually constrained RCM, [Guthart2000], a mechanically constrained RCM is often considered safer.

There is a variety of ways to achieve a mechanically constrained RCM [Turner2005], [Hamlin1994], [Vischer2000], [Baumann1997]. For those spherical mechanisms consisting of revolute joints, all the rotation axes intersect at the centre of the mechanism, effectively meeting the pivot constraint of MIS. The implementation of this approach can be done using parallelogram linkages [Rosen2002], [Taylor1995], or other linkage configuration [Guerrouad1989], [Lum2004]. However, in most cases, actuators are directly mounted at the joints. This may result in: (1) addition of inertial loads that adversely affect system performance [Berkelman2003], [Berkelman2006] or (2) lack of DOEs to place and guide the MIS instruments within the abdominal cavity [Lum2004], [Lum2006].

List of Literature References:
[Sackier1994] Sackier J, Wang Y. 1994. Springer; Robotically assisted laparoscopic surgery. Surgical endoscopy 8(1):63-66.
[Cavusoglu1999] Cavusoglu M, Tendick F, Cohn M, Sastry S. 1999. A laparoscopic telesurgical workstation. IEEE Transactions on Robotics and automation 15(4):728-739.
[Guthart2000] Guthart G, Salisbury K. 2000. The inituitive telesurgical system: Overview and application. Proceedings of the 2000 IEEE ICRA pp. 618-621.
[Turner2005] Turner M, Perkins D, Murray A, Larochelle P. 2005. Systematic Process for Constructing Spherical Four—Bar Mechanisms. ASME International Mechanical Engineering Congress and Exposition.
[Hamlin1994] Hamlin G J, Sanderson A C. 1994. San Diego, Calif.: A Novel Concentric Multilink Spherical Joint With Parallel Robotics Applications. IEEE International Conference on Robotics and Automation 2:1267-1272.
[Vischer2000] Vischer P, Clavel R. 2000. Multimedia Archives; Argos: a novel 3-DoF parallel wrist mechanism. The International Journal of Robotics Research 19(1):5.
[Baunnann1997] Baumann R. 1997. Haptic interface for virtual reality based laparoscopic surgery training environment. Unpublished doctoral dissertation 1734.
[Rosen2002] Rosen J, Brown J, Chang L, Barreca M, Sinanan M, Hannaford B. 2002. The bluedragon-a system for measuring the kinematics and the dynamics of minimally invasive surgical tools in-vivo. Proceedings- IEEE International Conference on Robotics and Automation 2:1876-1881.
[Taylor1995] Taylor R, Funda J, Eldridge B, Gomory S, Gruben K, LaRose D, Talamini M, Kavoussi L, Anderson J. 1995. A telerobotic assistant for laparoscopic surgery. IEEE Engineering in Medicine and Biology Magazine 14(3):279-288.
[Guerrouad 1989] Guerrouad A, Vidal P. 1989. SMOS: Stereotaxical microtelemanipulator for ocular surgery. Engineering in Medicine and Biology Society, 1989. Images of the Twenty-First Century., Proceedings of the Annual International Conference of the IEEE Engineering pp. 879-880.
[Lum2004] Lum M, Rosen J, Sinanan M, Hannaford B. 2004. Kinematic optimization of a spherical mechanism for a minimally invasive surgical robot. IEEE International Conference on Robotics and Automation 1:829-834.
[Lum2006] Lum M, Rosen J, Sinanan M, Hannaford B. 2006. Citeseer; Optimization of a spherical mechanism for a minimally invasive surgical robot: theoretical and experimental approaches. IEEE Transactions on Biomedical Engineering 53(7):1440-1445.
[Berkelman2003] Berkelman P, Boidard E, Cinquin P, Troccaz J. 2003. LER: The light endoscope robot. 2003 IEEE/RSJ International Conference on Intelligent Robots and Systems, 2003.(IROS 2003). Proceedings 3.

Other prior art documents include the following publications: US 2005/0096502, US 2009/0247821, GB 969,899, JP 2008-104620, U.S. Pat. No. 6,197,017, US 2002/0049367, US 2003/0208186, US 2005/0240078, US 2006/0183975, US 2007/0299387, EP 0 595 291, U.S. Pat. No. 6,233,504, US 2004/0236316, US 2004/0253079, US 2008/0058776, US 2008/0314181, US 2009/0198253, WO 03/067341, WO 2004/052171, WO 2005/046500, WO 2007/133065, WO 2008/130235, WO 03/086219, WO 2010/030114, DE 10314827, JP 2004041580, WO 2010/050771, WO 2010/019001, WO 2009/157719, WO 2009/145572, WO 2010/096580, DE 10314828, WO 2010/083480, U.S. Pat. No. 5,599,151, EP 1 254 642, CN 101584594, CN 101732093, U.S. Pat. No. 5,810,716, DE 4303311, US 2008/071208, US 2006/253109, WO 2009/095893, WO 2005/009482, CN 101637402, EP 0 621 009, WO 2009/091497, WO 2006/086663, EP 2 058 090.

### SUMMARY OF THE INVENTION

An aim of the present invention is to improve the known devices and methods.

More specifically, an aim of the present invention is to provide a novel external positioning manipulator able to provide sufficient dexterity and precision to position the MIS instruments. The unique design of the proposed system permits to keep the above mentioned characteristics at any location within the abdominal cavity. Extensive discussions with the surgical community have provided a precious input to establish a highly innovative engineering surgical system.

According to an embodiment of the present invention, a mechanical system for supporting and manipulating a terminal element in a surgical instrument may comprise:
a. at least one base platform; and
b. at least two moving platforms, a distal and a proximal one, able to move with three translational degrees of freedom and fixed orientation; and
c. at least one moving output link, the instrument shaft, whose distal extremity coincides with the end-effector of the said manipulator, whose proximal end is connected by a spherical joint to the said distal moving platform and its intermediate portion is connected to the said proximal moving platform by a slider spherical joint; and
d. at least three moving and identical limbs, each one consisting of an input link, directly connected to the said base platform, and two driven links, connecting the two said movable platforms to the said input link, where:
   i. this connections, between the different links, the three moving members and the base member, are done through mechanical articulations
   ii. these mechanical articulations have one degree of freedom between the base platform and the input links, at least two degrees of freedom between the input links and the driven links, at least two degrees of freedom between the driven links and the platform movable members, three degrees of freedom between the distal movable platform and the output link and at least three degrees of freedom between the distal movable platform and the output link
e. a remote centre of rotation, placed in the plane of the base platform, around which the output link can have three rotational degrees of freedom plus and translational degree of freedom in the direction of the output link axis
f. means of fixing in space the position and orientation of the end-effector and output link with respect to the base member, within the systems workspace
g. means of moving in space the position and orientation of the end-effector and output link with respect to the base member, within the systems workspace
h. an offset extension of the three platforms for a better positioning of the remote center of rotation
i. where the values geometry lengths and angles respect the Intercept theorem
j. said manipulator further comprising means for carrying said surgical instruments,

In one embodiment, at least one of the driven links may comprise two parallel bars, each of said bars being mounted at a first end by an articulation to an input link and at the other end by an articulation to one moving platform.

In one embodiment, each input link may be connected by a one-degree-of-freedom rotational articulation to the base frame, with the axis of that rotation belonging to the base plane.

In one embodiment, at least one of the driven links may comprise a single bar being mounted at the first end by a two-degrees-of-freedom cardan articulation to an input link and at the other end by a second two-degrees-of-freedom cardan articulation to one moving platform.

In one embodiment, the surgical instrument may be an endoscopic tool.

In one embodiment, the degrees of freedom may be controlled by active elements.

In one embodiment, the degrees of freedom may be controlled by passive elements.

In one embodiment, the active elements may be actuators (linear, rotational, electric, pneumatic, etc

In one embodiment, the passive elements may be brakes and/or clutches and/or springs and/or dampers.

In one embodiment, a surgical system may comprise a manipulator and a surgical table, wherein the manipulator has a predetermined position with respect to the table.

In one embodiment, in the surgical system, the manipulator may be fixed to the table, or to the floor or to another structure.

In one embodiment, the manipulator may be placed approximately in a plane defined by the table.

In one embodiment, the manipulator may be in a plane approximately perpendicular to a plane defined by the table.

In one embodiment, the manipulator may be in a plane that is between the plane defined by the table and a plane perpendicular to the plane defined by the table.

In one embodiment, the fixed platform may be movable and the distal moving platform is fixed.

These and other embodiments will be defined in the following description of the invention.

### DETAILED DESCRIPTION OF THE INVENTION

The present invention will be better understood by a detailed description of several embodiments therefrom and by reference to the following drawings in which

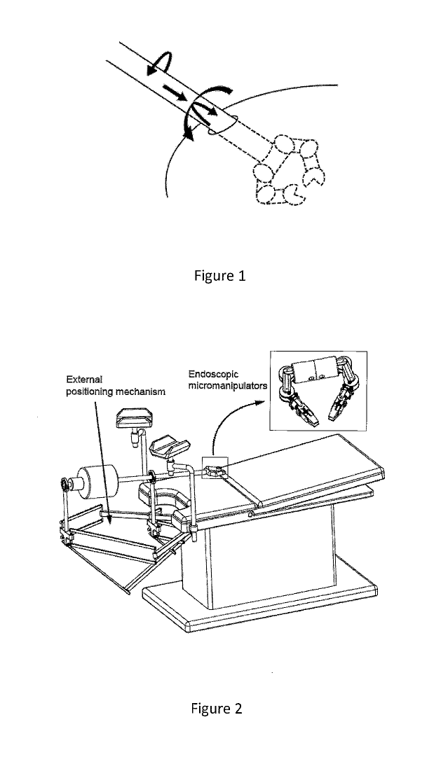

FIG. 1 illustrates a conceptual representation of a surgical platform;

FIG. 2 illustrates a conceptual design of the complete surgical platform;

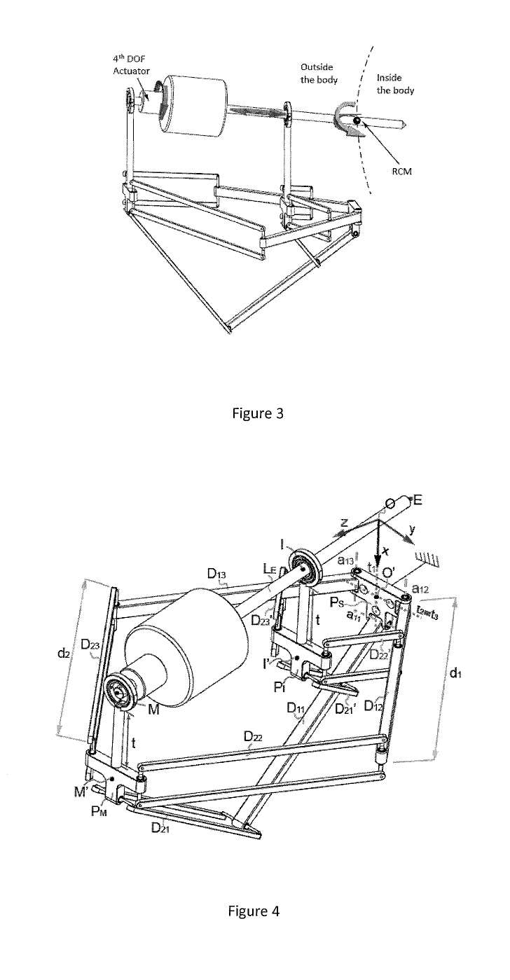

FIG. 3 illustrates degrees of freedom of an external manipulator;

FIG. 4 illustrates external manipulator schematics;

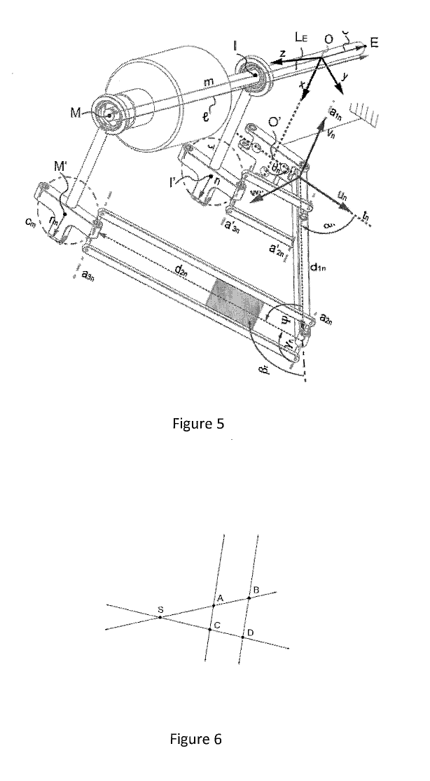

FIG. 5 illustrates limb schematics;

FIG. 6 illustrates the Intercept Theorem;

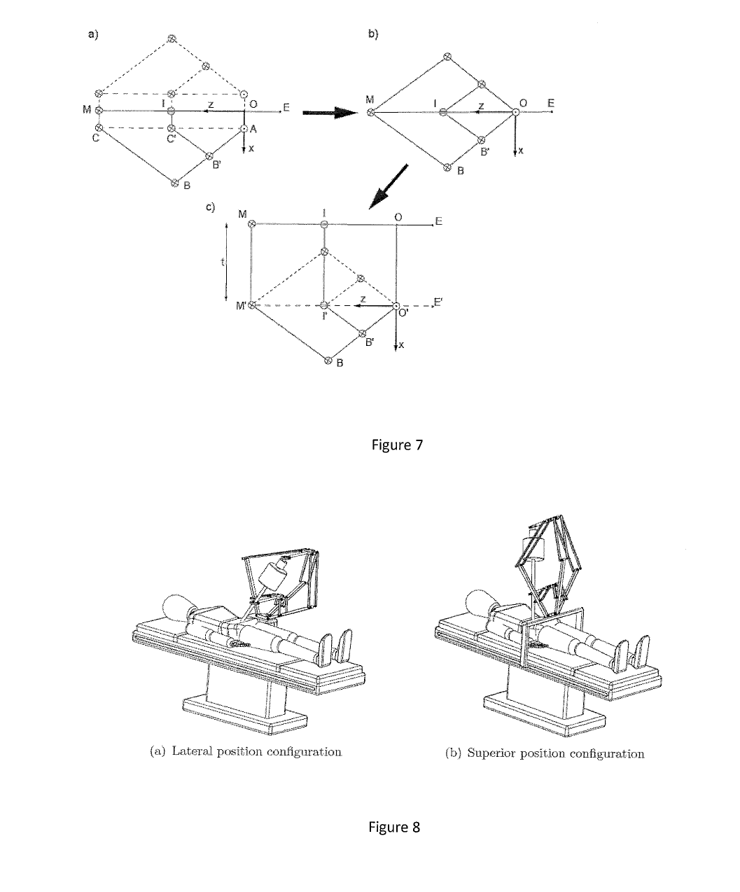

FIG. 7 illustrates a 2D representation of manipulator kinematics;

FIG. 8 illustrates examples of potential working configurations for the external manipulator;

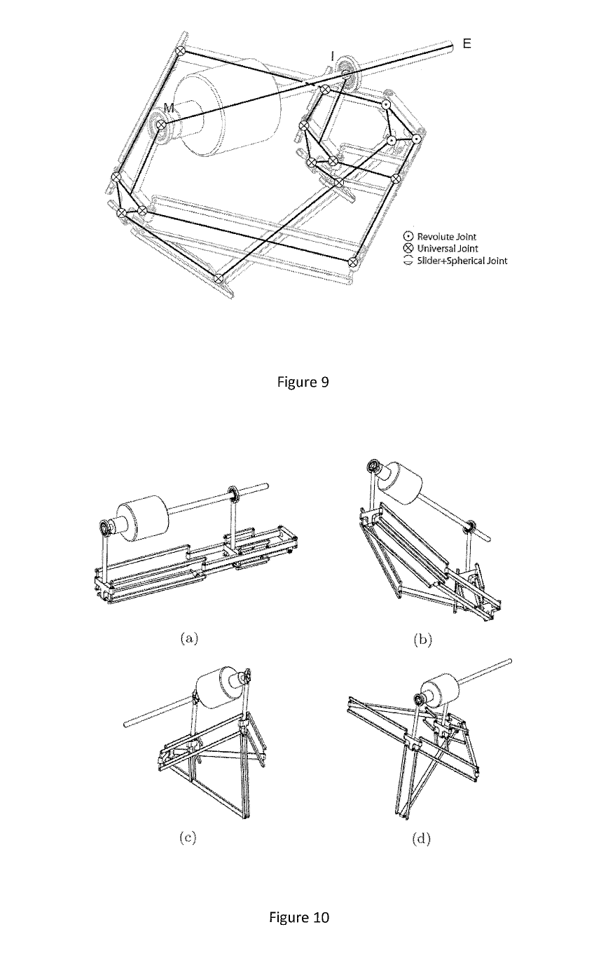

FIG. 9 illustrates a kinematic structure of the external manipulator

FIG. 10 illustrates examples of singular configurations;

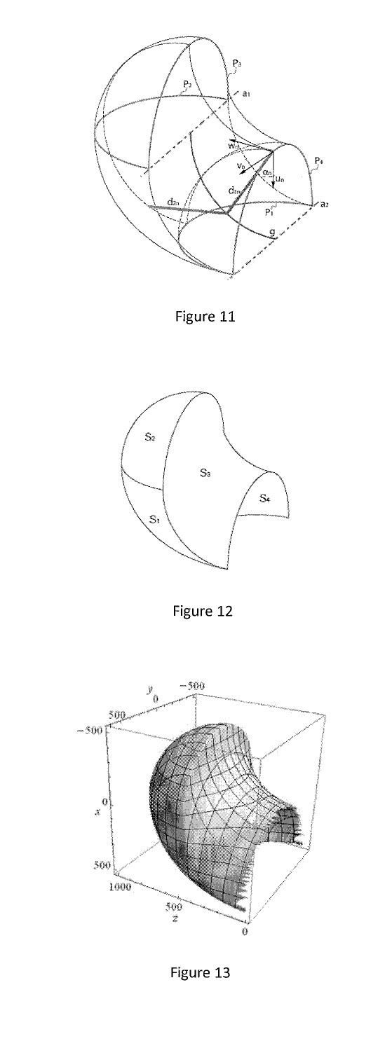

FIG. 11 illustrates profiles generating the nth limb workspace;

FIG. 12 illustrates workspace surfaces for each limb;

FIG. 13 illustrates a 3D representation of the workspace of point M, for a single limb (example with d=500 and I=1000 is shown);

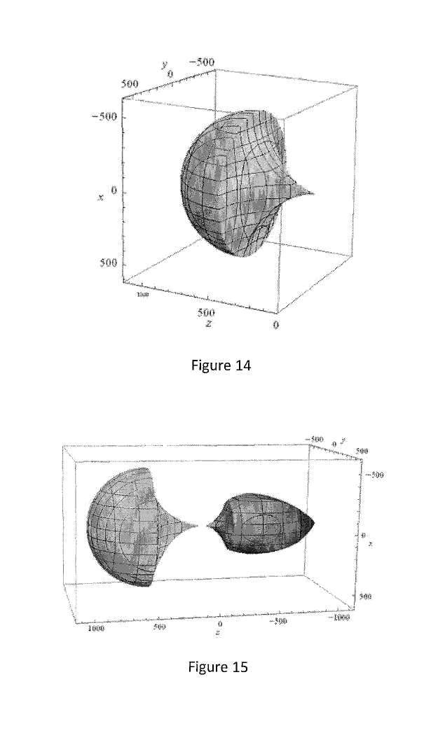

FIG. 14 illustrates a 3D representation of the workspace of point M (example with α₁=0 rad, α₂=π/2 and α₃=π/2 rad);

FIG. 15 illustrates a 3D representation of the workspace of points M (z>0) and E (z<0);

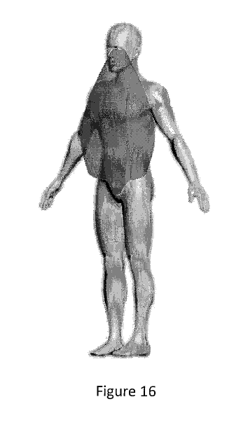

FIG. 16 illustrates a workspace with respect to patient.

The size and reduced dexterity of current surgical robotic systems are factors that restrict their effective performance. To improve the usefulness of surgical robots in minimally invasive surgery (MIS), a compact and accurate positioning mechanism is proposed in this paper. This spatial hybrid mechanism based on a novel parallel kinematics is able to provide three rotations and one translation. The corresponding axes intersect at a remote centre of rotation (RCM) that is the MIS entry port. Another important feature of the proposed positioning manipulator is that it can be placed below the operating table plane, allowing a quick and direct access to the patient, without removing the robotic system. This, besides saving precious space in the operating room, will significantly improve safety over existing solutions. The conceptual design of the system is presented in this document. Solutions for the inverse and direct kinematics are developed, as well as the analytical workspace and singularity analysis. The proposed manipulator design will contribute to increase the precision and stability of abdominal surgical procedures, increasing their reliability. This is possible taking into account the performance of the presented parallel structure.

The idea beyond this invention consists in bringing precise manipulation and standard surgical procedures inside the abdominal cavity, with a remotely actuated micro-robotic system, stabilized by an external system and inserted through an incision on the supra-pubic hair region, see FIG. 1 that shows a conceptual representation of the surgical platform.

The surgical platform proposed, illustrated in FIG. 2 mainly comprises two subsystems: (1) an external positioning unit and (2) an endoscopic unit. A micro-manipulator system operates to increase the degree of dexterity, payload capacity, stiffness and precision inside the patient's body.

The purpose of the external manipulator is to position the micro-manipulators of endoscopic units inside the human body, without violating the constraints imposed by the fixed tissue incision point. In this way, the proposed external manipulator provides 4 DOF, with a fixed RCM, for positioning endoscopic micromanipulators inside the abdominal cavity. The related kinematics gives to the insertion tube (IT) two rotary degrees of freedom about the incision port plus a linear movement in the direction of the same point, along the axis of the IT. The fourth DOF is a rotation about the IT's axis, given by a fourth degree of freedom of the external unit, see the degrees of freedom of the external manipulator illustrated in FIG. 3.

Since the external manipulator cannot provide the desired mobility needed to perform complicated surgical procedures, the extra DOFs are given by endoscopic micro-manipulators.

Despite showing good operating characteristics (large workspace, high flexibility and dexterity), serial manipulators present disadvantages, such as low precision, low stiffness and low payload. On the other hand, parallel kinematic manipulators offer essential advantages, mainly related t o lower moving masses, higher rigidity and payload-to-weight ratio, higher natural frequencies, better accuracy, simpler modular mechanical construction and possibility to locate actuators on the fixed base. These characteristics make parallel manipulators extremely suitable for surgical applications. Taking into account that stiffness and precision are considered to be key features on external positioning mechanisms for MIS, the proposed manipulator is based on a parallel kinematics, to reproduce the needed degrees of freedom.

A schematic of the proposed manipulator is shown in FIG. 4. The RCM, point O, is placed on the X-axis of the fixed reference frame, F(x, y, z), and is distant by an offset t from the origin, O′, which is placed in the intersection of lines t₁, t₂ and t₃, that belong to the stationary platform, P_{S}, in the XY plane. In addition, lines t₁, t₂ and t₃ are perpendicular to axes a₁₁, a₁₂ a₁₃, respectively. Three identical limbs connect the moving platforms, P_{M} and P_{I}, to the stationary platform. Each limb consists of an input link, directly connected to the actuator, placed on P_{S} and two driven links, connected to P_{M} and P₁. The input links are labelled D₁₁, D₁₂, and D₁₃ and have length d₁. The driven links are composed by planar four-bar parallelograms, D₂₁, D₂₂, D₂₃, D₂₁, D′₂₂ and D′₂₃ and have length d₂ and d′₂ respectively. All of the links and platforms are considered as rigid bodies (FIG. 4).

The n^{th} limb of the manipulator is shown in FIG. 5. In each limb, the driven links, the input link, and the three platforms are connected by four parallel revolute joints, at axes a₁ₙ, a₂ₙ, a₃ₙ, and a′₃ₙ that are perpendicular to the axes of the four-bar parallelogram for each limb. A coordinate system, Lₙ(uₙ,vₙ,wₙ), is attached to the fixed base, P_{S}, in the actuated joint of each limb, such as the uₙ axis is perpendicular to the axis of rotation of the joint, a₁ₙ, and at an angle θₙ from the x-axis, while being in the plane of P_{S}. The vₙ-axis is along a₁ₙ.

The actuation angle, αₙ, for the n^{th} limb, defines the angular orientation of the input link relative to the XY plane, on platform P_{S}. Vectors m and e are respectively the position vectors of points M and E, in the F coordinate frame. M and I are placed at the centre of circles c_{M} and c_{I} of radius r_{M} and r_{I}, that belong to platforms P_{M} and P_{I}. Vector I is aligned with the output link, L_{E}, from point M to point E. Angles βₙ and β′ₙ are defined from the direction of input links, axis d₁ₙ, to the direction of the plane containing the parallelograms of driven links, d₂ₙ and d′₂ₙ. Angles γn and γ′ₙ are defined by the angles from the directions of the driven links, d₂ₙ and d′₂ₙ, through axis a₂ₙ and a′₂ₙ.

The configuration of the limbs is based on the well known Delta robot. It is in fact composed by a pair of 3 four-bar-parallelogram-links fixed on the same input links. Therefore, the two platforms (the intermediate, P_{I}, and the distal one, P_{M}) move in the same manner except that P_{M} moves with bigger ranges than P_{I}. Link, L_{E}, containing the end-effector, E, is then connected to points M and I by an universal joint and a sliding spherical joint respectively. The output of the proposed design results in: two rotations of L_{E} around the X and Y axis, and a translation of E on the direction MO.

To guarantee a perfect RCM, a geometrical ratio is needed. This ratio is based on the Intercept Theorem, which states that: if two or more parallel lines are intersected by two self intersecting lines, then the ratios of the line segments of the first intersecting line is equal to the ratio of the similar line segments of the second intersecting line. In other words, and for the example of FIG. 6:

$$\frac{\overline{SD}}{\overline{CD}} = \frac{\overline{SB}}{\overline{AB}}$$

On FIG. 7(a), a simplified 2D representation of the Manipulator is shown. The upper limb (“dashed”) is virtually rotated ir rad from the one below, around the Z axis. According to the Delta principle, the rotations of the moving platforms are blocked and P_{M} and P_{I} are always parallel and vertical. Consequently, in order to have the link ME always aiming at the RCM, it is necessary to have points A, C′ and C aligned. This is true if segments B′C′ and BC are parallel and if BC/B′C′=AB/AB′ (Intercept Theorem). If these conditions are not fulfilled, the behavior of the robot will be similar but without a perfect RCM. By contrast, if they are satisfied, point I will always be aligned with O and M, for any position of M, and platform P_{I} is passively moved to guarantee this configuration. According to the above mentioned constraints, a geometrical simplification can be made, assuming zero-size platforms, which significantly simplifies the kinematic analysis of the mechanical structure, FIG. 7 (b). In addition, an equivalent architecture can be introduced, extending the platforms at O, I and M by a distance t, as shown in FIG. 7(c). In this way, the RCM is translated by a distance t, in the platform's extension direction, resulting in a mechanism with the same kinematics.

It is also important to point out that this kinematics can also be applied in other configurations specific to different surgical procedures. FIG. 8 shows two other possible configurations of the proposed kinematics.

Manipulator Mobility

The proposed parallel platform hereafter is characterized by the kinematic structure shown in FIG. 9. Considering the manipulator mobility, let F be the degrees of freedom, n the number of parts, k the number of articulations, fᵢ the degrees of freedom associated with the i^{th} joint, and λ=6, the motion parameter. Then, the number of DOFs of a mechanism is determined by the Grübler-Kutzbach Criterion:

F=γ(n−k)+Σᵢ₌₁^{j}fᵢ=6(13−18)+33=3

For this manipulator, we have: n=13 (3 inputs links, 6 driven links, 2 moving platforms, 1 slider-mount, 1 end-effector link); k=18 (3 actuated revolute joints, 1 spherical joint, 13 universal joints and 1 slider) and Σfᵢ=33. Applying the equation above to the External Manipulator results in: F=3, and consequently a mechanism with 3 DOF. The result would be the same considering all the bars of the parallelograms with a ball and a universal joint at each tip.

The kinematics of Delta-like Manipulators has been extensively studied by several authors. Although they look similar in form, this manipulator kinematics is simpler due to the dimensional constraints imposed by the Intercept Theorem as well as by the geometrically equivalent zero-sized platforms simplification (represented in FIG. 7). Although the RCM might not be completely stationary in a real prototype, due to a deficient production of the different components, in the following analysis it is assumed so, with the Intercept Theorem constraints perfectly fulfilled.

For the inverse geometrical model, the objective is to find the set of joint angles, (α₁, α₂, α₃), that achieve a certain position of the end-effector, E(eₓ,e_{y},e_{z}) in the F(x,y,z) coordinate system. Considering the geometry of the manipulator, shown in FIG. 5, it is possible to write the following relations for each limb:

d₁ₙ+d₂ₙ=m=e−1

where I is the vector going from point M to point E and:

$${}^{F} e = [ \begin{matrix}{e}_{x} \\ {e}_{y} \\ {e}_{z}\end{matrix} ] , \\ {}^{F} l = - \frac{e}{‖ e ‖} ‖ l ‖ , \\ {}_{}^{F}{d}{}_{1 n}^{} + {}_{}^{F}{d}{}_{2 n}^{} = {}_{}^{F}{R}{}_{L}^{} ( {}_{}^{{L}_{n}}{d}{}_{1 n}^{} + {}_{}^{{L}_{n}}{d}{}_{2 n}^{} ) , \\ {}_{}^{F}{R}{}_{L}^{} = [ \begin{matrix}cos {θ}_{n} & - sin {θ}_{n} & 0 \\ sin {θ}_{n} & cos {θ}_{n} & 0 \\ 0 & 0 & 1\end{matrix} ] , \\ {}_{}^{{L}_{n}}{d}{}_{1 n}^{} + {}_{}^{{L}_{n}}{d}{}_{2 n}^{} = [ \begin{matrix}{d}_{1} cos {α}_{n} + {d}_{2} sin {γ}_{n} cos ( {α}_{n} + {β}_{n} ) \\ {d}_{2} cos {γ}_{n} \\ {d}_{1} sin {α}_{n} + {d}_{2} sin {γ}_{n} sin ( {α}_{n} + {β}_{n} )\end{matrix} ] ,$$

Expanding those relations in the Lₙ(uₙ,vₙ,wₙ) coordinate frame, the analytical expressions of αₙ, βₙ and γₙ, for the three limbs, can be obtained.

The Direct Geometrical Model describes the position of the end-effector, E(eₓ,e_{y},e_{z}), given a set of known actuated joint angles, (α₁, α₂, α₃), in the F(x,y,z) coordinate frame.

Given its special kinematics, the first step to solve the direct geometric model of this manipulator consists in finding the solutions for point M. The surface of each sphere represents the range of motion of distal end of the n^{th} limb, when point Bₙ is located at a known position. The radius of each sphere is equivalent to length d₂ and the intersection points of the three sphere surfaces are the possible positions that point M may occupy. The equation of the sphere generated by the n^{th} limb is given by:

(mₓ−bₙₓ)²+(m_{y}−b_{ny})²+(m_{z}−b_{nz})²=d₂²

Finally, after calculating the coordinates of point M, the end-effector coordinates are obtained by:

$$e = - \frac{m}{‖ m ‖} ( ‖ l ‖ - ‖ m ‖ )$$

which solves the direct kinematics problem for this manipulator.

Due to the relatively high complexity of the inverse kinematics equations for this manipulator, it is not computationally efficient to calculate the Jacobian Matrix, differentiating those relationships with respect to x, y and z. As an alternative, the velocity of the end-effector, v_{E} is obtained by differentiating the equation of the limb geometrical constraints with respect to time:

$${v}_{E} = {v}_{M} - ( \frac{m}{‖ m ‖} ‖ l ‖ ) ,$$

which, after some expansion, results in three scalar equations that can be arranged as follows:

Jₓv_{M}=J_{q}\dot{q}

where the direct and inverse kinematics Jacobian matrices are respectively:

$${J}_{x} = [ \begin{matrix}{j}_{1}^{T} \\ {j}_{2}^{T} \\ {j}_{3}^{T}\end{matrix} ] , \\ {J}_{q} = [ \begin{matrix}{b}_{1} & 0 & 0 \\ 0 & {b}_{2} & 0 \\ 0 & 0 & {b}_{3}\end{matrix} ]$$

$$with \text{:}$$

$${j}_{n} = [ \begin{matrix}cos ( {α}_{n} + {β}_{n} ) sin {γ}_{n} cos {θ}_{n} - cos {γ}_{n} sin {θ}_{n} \\ cos ( {α}_{n} + {β}_{n} ) sin {γ}_{n} cos {θ}_{n} - cos {γ}_{n} sin {θ}_{n} \\ cos ( {α}_{n} + {β}_{n} ) sin {γ}_{n}\end{matrix} ] , for n = 1 , 2 , 3$$

$${b}_{n} = a sin {β}_{n} sin {γ}_{n} , for n = 1 , 2 , 3$$

$$and$$

$$q = [ \begin{matrix}{\dot{α}}_{1} \\ {\dot{α}}_{2} \\ {\dot{α}}_{3}\end{matrix} ]$$

The identification of singular configurations is an important issue that must be addressed at the first stages of mechanisms design. This topic has been studied for a long time and comprehensive classifications have been proposed in past years. The most remarkable cases are usually called (1) inverse kinematics singularities, when an infinitesimal motion of a limb does not yield a motion of the platform (that “loses” one or more DOF in certain directions) and (2) direct kinematics singularities, when the moving platform can move along certain directions even if all actuators are completely locked (and the mechanism “gains” one or more DOF). From the previous section:

v_{E}=f(m, v_{M})

Which can be simplified by:

v_{E}=f(m,Jₓ⁻¹J_{q}\dot{q})

To summarize, singularities can occur when:
all the pairs of the bars composing the parallelograms are parallel - the moving platforms have three degrees of freedom and move along a spherical surface rotating about an axis perpendicular to the platforms, FIG. 10(a).
two pairs of bars composing the parallelograms, for each moving platform, are parallel—the moving platforms have one degree of freedom, moving in only one direction FIG. 10(b).
two pairs of bars composing the parallelograms are in the same plane or in parallel planes—the moving platforms have only one degree of freedom, rotating about a vertical axis, FIG. 10(c).
three parallelograms, of each moving platform, are placed at three parallel planes or on the same plane—the platforms keep three DOFs, namely: two rotations about axes contained in the plane of the platform and one translation perpendicular to the same plane, FIG. 10(d).

Workspace is one of the most important issues when designing a parallel manipulator since it determines the region that can be reached and, therefore, it is a key point in robotic mechanism design. The designs based on a workspace calculation use methods in which the first step is to develop an objective function that might be reached by the result. The result is generally obtained by recursive-numerical-algorithms. These methodologies have the disadvantage of being extremely time consuming, due to the highly non-linear objective functions that are manipulated. In this paper, the workspace representation of this manipulator is analyzed geometrically.

Knowing in advance all the singular configurations presented in the previous section, it is possible to introduce some constraints in the manipulator's design in order to avoid those postures and collisions between mechanism components. Therefore, it was decided to analyze the workspace of the manipulator in the boundary of those conditions, where αₙ ∈ [0, π/2], γₙ ∈ [0, π] and d₁=d₂=d. For a given position of the moving point M, the position of the end-effector, E, can be determined by a translation through vector I. In other words, the workspace generated by the n^{th} limb is a translation of the reachable workspace of point M by I. In addition, the motion of the limb is constrained, not only by the joint limits, but also by the other limbs. Therefore, the workspace of this manipulator is the intersection of the three individual reachable workspaces generated by the three limbs.

According to the specific limb design, the workspace of the limb point M is a solid sphere with radius d, if there are no joint limitations for the revolute joints. However, point Bₙ (bₙₓ, b_{ny}, b_{nz}), which is able to move along a circular path in the ZX plan, is limited to avoid singular configurations and collisions with other components of the mechanism. The workspace of each limb is the solid envelope shown in FIG. 11:

With the profiles presented before, it is possible to generate the surfaces shown in FIG. 12.

Once the former analytical expressions have been identified, it is possible to represent them in the 3D space, using Wolfram Mathematica 7, and visualize the workspace of the manipulator, as in FIG. 13.

The workspace of M, considering the entire manipulator, is the result of the intersection of the workspaces of the 3 limb workspaces, see FIG. 14.

Having the Workspace of point M, W_{M}, defined, the workspace of E, W_{E}, is calculated using Eq. 13, see FIG. 15. On the left part of the plot, for z>0, we may find the workspace of M, while the workspace of E is represented for z<0. As can be seen by the workspace distribution around point O (0,0,0), the stationary of the mechanism's RCM is verified.

The reachable workspace of the Manipulator can be represented easily using the commercial CAD software such as Solid Works 2009. For instance, in the example with d=500, I=1000, α₁=0 rad, α₂=π/2 and α₃=−π/2 rad, the workspace has the shape shown on FIG. 16. It can be seen that it encloses the patients abdominal cavity, meeting the specifications in terms of task workspace, for MIS.

The examples given above are only illustrative and should not be interpreted or understood in a limiting manner on the scope of the present invention and claims. Variants are possible within the scope and frame of the present invention, for example by using equivalent means.

Also, the different embodiments described in the present application may be combined together as desired by the user.

1. An external manipulator for positioning surgical instruments,
said manipulator comprising an hybrid kinematic with a parallel structure able to provide three positional degrees of freedom with a remote centre of rotation, said degrees of freedom being two rotations and one translation, and a fourth orientation degree of freedom, whose axis crosses said remote centre of rotation,
said manipulator further comprising means for carrying said surgical instruments,
such that said surgical instrument can be displaced and oriented about said remote centre of rotation by said manipulator.

2. A manipulator as defined in claim 1, wherein said means comprise an insertion tube.

3. A manipulator as defined in claim 1, wherein said surgical instrument is an endoscopic tool.

4. A manipulator as defined in claim 1, wherein said degrees of freedom are controlled by active elements.

5. A manipulator as defined in claim 1, wherein said degrees of freedom are controlled by passive elements.

6. A manipulator as defined in claim 1, wherein the active elements are actuators.

7. A manipulator as defined in claim 1, wherein the passive elements are brakes and/or clutches and/or springs and/or dampers.

8. A surgical system comprising a manipulator as defined in claim 1 and a surgical table, wherein the manipulator has a predetermined position with respect to the table.

9. A surgical system as defined in claim 8, wherein the manipulator is fixed to the table, or to the floor or to another structure.

10. A surgical system as defined in claim 8, wherein said manipulator is placed approximately in a plane defined by said table.

11. A surgical system as defined in claim 8, wherein said manipulator is in a plane approximately perpendicular to a plane defined by said table.

12. A surgical system as defined in claim 8, wherein said manipulator is in a plane that is between the plane defined by the table and a plane perpendicular to the plane defined by the table.
